(19) United States
(12) Patent Application Publication
(10) Pub. No.: US 2007/0087349 A1
(43) Pub. Date: Apr. 19, 2007

(54) Highly parallel template-based dna synthesizer

(75) Inventors: Peer Staehler, Mannheim (DE); Cord Stahler, Weinheim (DE); Markus Beier, Heidelberg (DE)

Correspondence Address: ROTHWELL, FIGG, ERNST & MANBECK, P.C., 1425 K STREET, N.W., SUITE 800, WASHINGTON, DC, 20005, US

(73) Assignee: Febit Biotech GmbH, Heidelberg (DE)

(21) Appl. No.: 10/579,769

(22) PCT Filed: Nov. 18, 2004
(86) PCT No.: PCT/EP04/13131

§ 371 Date: Jul. 24, 2006

(30) Foreign Application Priority Data
Nov. 18, 2003 (DE) 103 53 887.9

(51) Int. Cl. C12Q 1/68; C12P 19/34; C12N 15/00

(52) U.S. Cl. 435/6; 435/455; 435/91.2

(57) ABSTRACT

The invention relates to a template-based method for preparing nucleic acid double strands.

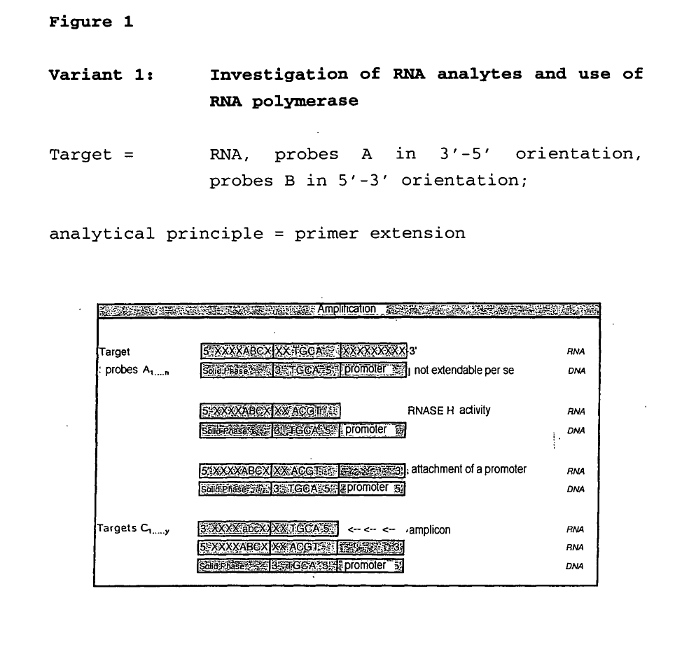

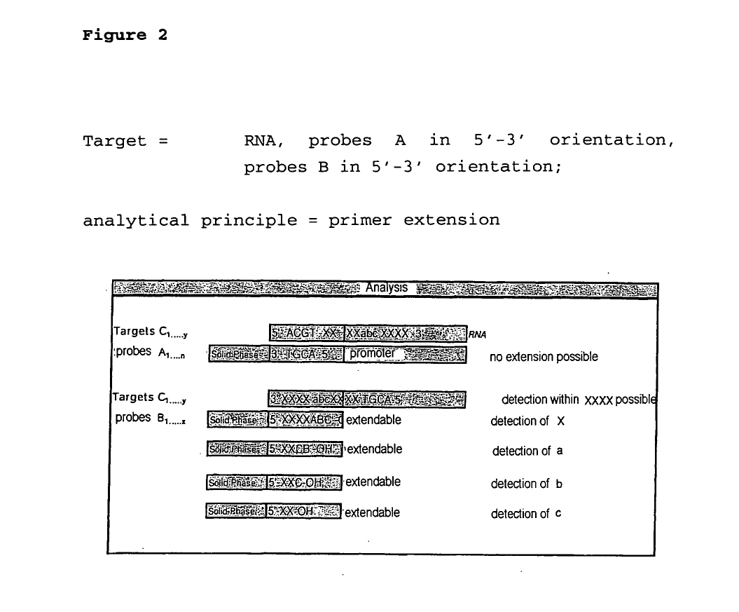

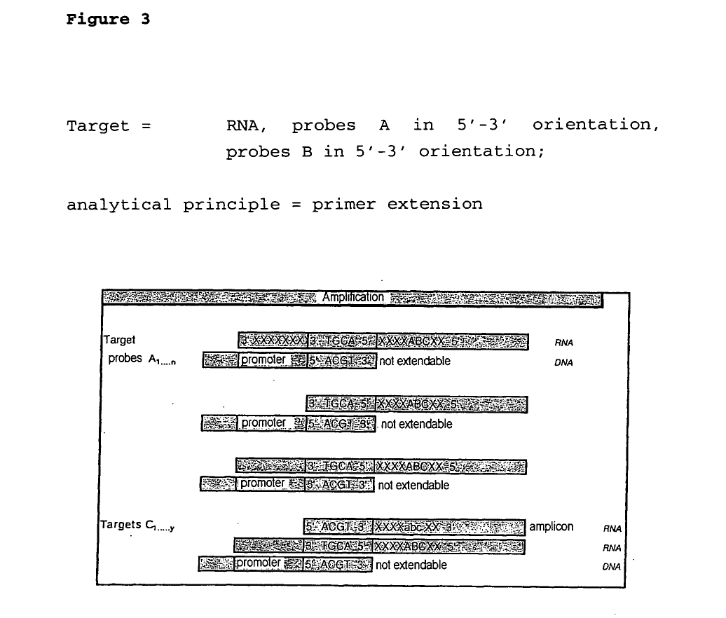

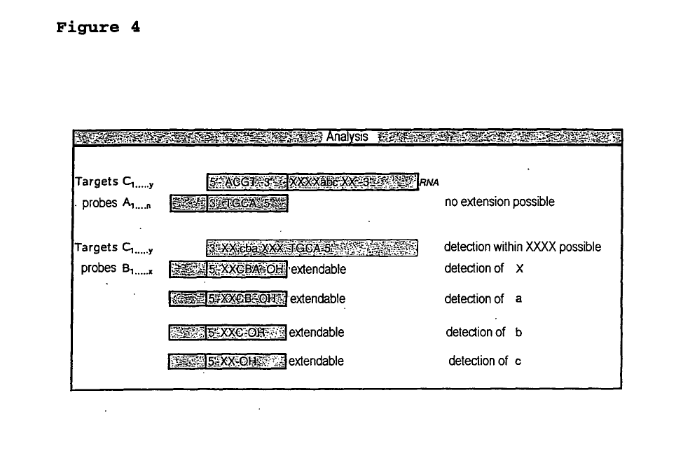

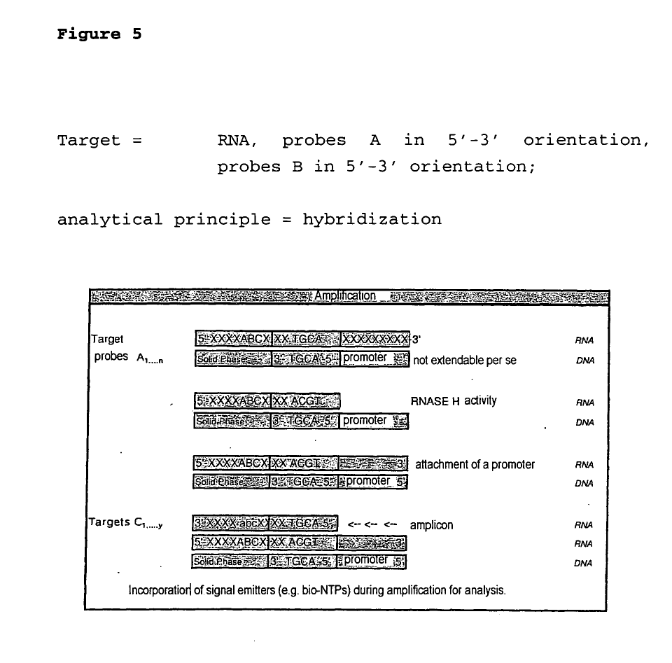

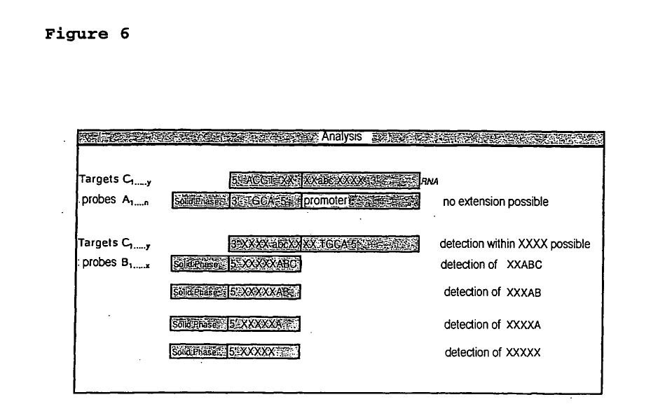

### 1.1 INTRODUCTION

The preparation of synthetic nucleic acids (DNA, RNA or their analogues) is mainly carried out with the aid of column-based synthesizers. The demand for such synthetic nucleic acids has increased greatly through molecular biology and biomedical research and development.

Particularly important and widespread areas of use of synthetic nucleic acid polymers are primers for the poymerase chain reaction (PCR) [Critical Reviews in Biochemistry and Molecular Biology 26 (3/4), pp. 301-334 , 1991] and the Sanger sequencing method [Proc. Nat. Acad. Sci. 74, pp. 5463-5467, 1977].

Synthetic DNA also plays a part in the preparation of synthetic genes [1. WO 00/13017 A2, 2. S. Rayner et al., PCR Methods and Applications 8 (7), pp. 741-747, 1998, 3. WO 90/00626 A1, 4. EP 385 410 A2, 5. WO 94/12632 A1, 6. WO 95/17413 A1, 7. EP 316 018 A2, 8. EP 022 242 A2, 9. L. E. Sindelar and J. M. Jaklevic, Nucl. Acids Res. 23 (6), pp. 982-987, 1995, 10. D. A. Lashkari, Proc. Nat. Acad. Sci. USA 92 (17), pp. 7912-7915, 1995, 11. WO 99/14318 A1].

Two newer fields of application with an increasing demand are the preparation of microarrays of oligonucleotide probes [1. Nature Genetics, Vol. 21, supplement (in full), Jan. 1999, 2. Nature Biotechnology, Vol. 16, pp. 981-983, Oct. 1998, 3. Trends in Biotechnology, Vol, 16, pp. 301-306, July 19989] and the preparation of interfering RNA (iRNA or RNAi) for modulating gene expression in target cells [PCT/EP01/13968].

Said areas of application of molecular biology provide valuable contributions in drug development, drug production, combinatorial biosynthesis (antibodies, effectors such as growth factors, neurotransmitters etc.), in biotechnology (e.g. enzyme design, pharming, biological preparation processes, bioreactors etc.), in molecular medicine, in the development and application of diagnostic aids (microarrays, receptors and antibodies, enzyme design etc.), or in environmental engineering (specialized or tailored microorganisms, production processes, remediation, sensors etc.). The method of the invention can thus be applied in all these areas.

### 1.2 PRIOR ART

The most widely used method for preparing synthetic nucleic acids is based on the fundamental work of Caruthers and is described as the phosphitamide method (M. H. Caruthers, Methods in Enzymology 154, pp. 287-313, 1987). The sequence of the resulting molecules can in this case be controlled by the synthetic sequence. Other methods such as, for example, the H-phosphonate method serve the same purpose of successive assembly of a polymer from its subunits, but have not become so widely used as the Caruthers method.

In order to be able to automate the chemical method of polymer synthesis from subunits, usually solid phases to which the growing molecular chain is tethered are used. It is eliminated only after the synthesis is complete, for which purpose a suitable linker between the actual polymer and the solid phase is necessary. For the automation, the method ordinarily uses solid phases in the form of activated particles which are packed into a column, e.g. controlled pore glass (CPG). Such solid phases ordinarily carry only one sequence type which can be separated and removed in a defined manner. The individual synthesis reagents are now added in a controllable manner in an automated device which ensures in particular automated addition of the individual reagents to the solid phase. The amount of synthesized molecules can be controlled through the amount of the support material and the size of the reaction mixtures. These amounts are either adequate or in fact too high (e.g. in the case of PCR primers) for the abovementioned molecular biology methods. A certain parallelization to generate a multiplicity of different sequences is achieved by arranging a plurality of columns in one apparatus construction. Thus, instruments with 96 parallel columns are known to the skilled worker.

One variant and further development of the preparation of synthetic nucleic acids is the in situ synthesis of microarrays (array disposition of the nucleic acids in a matrix). This is carried out on a substrate which is loaded by the synthesis with a multiplicity of different sequences. Detachment of the synthetic products is not provided in this case. The great advantage of in situ synthetic methods for microarrays is the provision of a multiplicity of molecules of differing and defined sequence at addressable locations on a common support. The synthesis in this case falls back on a limited set of starting materials (in the case of DNA microarrays ordinarily the 4 bases A, G, T and C) and assembles therefrom any desired sequences of nucleic acid polymers.

Segregation of the individual molecular species can take place on the one hand by separate fluidic compartments in the addition of the synthesis starting materials, as is the case for example in the so-called in situ spotting method or piezoelectric techniques, based on the inkjet printing technique (A. Blanchard, in Genetic Engineering, Principles and Methods, Vol. 20, Ed. J. Sedlow, pp. 111-124, Plenum Press; A. P. Blanchard, R. J. Kaiser, L. E. Hood, High-Density Oligonucleotide Arrays, Biosens. & Bioelectronics 11, pp. 687, 1996).

An alternative method is the spatially resolved activation of synthesis sites, which is possible for example through selective illumination or selective addition of activating reagents (deprotection reagents). The amount of synthesized molecules of a species is comparatively small in the methods disclosed to date, because by definition only small reaction zones are provided respectively for each sequence in a microarray, in order to be able to copy as many sequences as possible in the array and thus for functional use.

Examples of methods disclosed to date are the photo-lithographic light-directed based synthesis [McGall, G. et al; J. Amer. Chem. Soc. 119; 5081-5090; 1997], the projector-based light-directed synthesis [PCT/EP99/06317], the fluidic synthesis with separation of reaction chambers, the indirect projector-based light-controlled synthesis using photo acids and suitable reaction chambers in a microfluidic reaction support, the electronically induced synthesis by means of spatially resolved deprotection at individual electrodes on the carrier and fluidic synthesis by means of spatially resolved deposition of the activated synthesis monomers.

The use of support-bound libraries is described for the synthesis of synthetic genes in PCT/EP00/01356. One disadvantage of this method is that the matrix of molecules is destroyed by the dissolving-out step. A second important point is the amount of synthetic DNA which can be prepared per reaction site in the support, i.e. per type of oligo. In addition, the small scale which is associated by definition with the synthesis makes it not easy to handle the dissolved-out oligonucleotides.

One use of support-bound nucleic acids which are prepared in an array arrangement is indicated on the home page of Dr. J. Hoheisel's research group at the Deutsche Krebsforschungszentrum Heidelberg (DKFZ). One topic here is, for example, the use of nucleic acids as PCR primers. However, in this case too detachment of the molecules directly from the support is described. A use as template is not described.

A further use of support-bound nucleic acids which are prepared in an array arrangement is described in Bulyk et al. [Bulyk, M. et al; Nat. Biotech. 17; 573-577; 1999]. In this application, microarrays are assembled by means of Affymetrix photolithographic light-directed synthesis in such a way that different 25-mers with free 5′ ends are prepared on the solid phase. These are then filled in to give double strands by proximally binding primers. These double-strand arrays are then used to analyze binding events with DNA-binding proteins. In addition, enzymatic digestions with restriction enzymes are described for analytical purposes. A use of the generated copies after separation from the oligonucleotides serving as templates is not described. Nor is repeated or cyclic copying described. Since the synthetic method used is based on photolithography, it is moreover evident to the skilled worker that considerable effort, including the creation of appropriate masks, is necessary for a new array design and new nucleic acid sequences.

The use of photolithographic technology is very suitable for accurate illumination of patterns during the synthesis. This makes routine parallel preparation of high-density arrays possible. However, this approach is subject to some restrictions because it requires physical instead of digital constructions. In particular, the preparation of mask sets is a costly and time-consuming process. In summary, although Bulyk et al. take the first steps toward adding to the single-stranded nucleic acid to give the double strand, they then only go in the direction of further analytical applications of this double-strand array (binding assays with DNA-binding proteins) and do not propose any other use of the generated copies after separation from the template, the repeated or cyclic preparation thereof or a combination with sample amplification, as are described as embodiments of the method of the invention hereinafter, nor do they make such an invention obvious.

Also known is the solid phase-based amplification of target nucleic acids, e.g. the pool of mRNA molecules of a biological extract [Linden Bioscience, publication on “Solid Phase Transcription Chain Reaction” or “SP-TCR”]. For this purpose, two different primers which comprise sequences for the viral RNA polymerase promoters T3 and T7, also elements for hybridization of poly-T RNA in conjunction with the T7 promoter primer, were coupled to a solid phase (an in situ synthesis is not described, nor is it obvious to the skilled worker in view of two primers). After hybridization of an mRNA over the poly-A region, the strand is filled in to give the double strand. Subsequently, a specific cassette (TCR adapter) which in turn has one recognition site in common with the T3 promoter primer is ligated to the double strand. This results in a transcription chain reaction. The SP-TCR method functions highly efficiently on the solid phase. The preparation, underlying the method of the invention, of a library of template nucleic acids as starting point for copying processes is not obvious.

In a likewise solid phase-based approach to amplification by strand displacement, Westin et al. [Westin L. et al.; Nat. Biotech 18; 199-204; 2000] showed that the parallel use of more than one primer on a common solid phase is possible with high efficiency. However, in Westin et al., the primer nucleic acids are prepared separately from the reaction support and not in situ and are sited thereon only subsequently. The central aspect of the highly efficient in situ synthesis is thus inapplicable. In addition, there is no hint to be found that only the copies of the template nucleic acids are the actual participants in the reaction. On the contrary, the other primers having the analyte-specific sequence are also prepared externally and then added to the reaction. A copy of the template is not carried out.

### 1.3 SUBJECT MATTER OF THE INVENTION

The intention is to provide a method for preparing a plurality of different synthetic nucleic acids of any chosen sequence by preparing suitable solid phase-based synthetic libraries as templates and a template-dependent biochemical copying reaction.

It is thus possible for nucleic acid strands to be copied in high yield and simultaneously with very many different sequences by a support with a library thereon and to be made available for further process steps.

The invention accordingly relates to a method for the enzyme-based synthesis of nucleic acids by copy of a template library synthesized as array in a matrix, carried out on an enzyme-based nucleic acid matrix synthesizer as apparatus.

Preferred embodiments of the invention are represented in claims 1 to 31.

### 1.4 OUTLINES OF THE SOLUTION ROUTE

The templates for the enzyme-based synthesis by means of a copying process consist in turn of copyable nucleic acid polymers which are synthesized in the form of an array arrangement on a common support. After their actual synthesis, they are available in a copyable state and can be amplified in an enzyme-based method with addition of appropriate reagents and aids such as nucleotides.

By using known methods for preparing such arrays of nucleic acid polymers, e.g. in the form of a so-called microarray, it is possible to generate very many (typically more than 10) different nucleic acid polymers with a length of at least more than 2, typically more than 10, bases.

Examples of such methods are described above. All these methods eventually lead to a library or a set of oligo-nucleotides or polynucleotides on a common support. The abovementioned concept of nucleic acid polymers in a matrix arrangement is intended to encompass this. All these methods serve essentially to prepare so-called microarrays for analyzing nucleic acids by means of hybridization.

The next step in the method of the invention now consists of copying, with the aid of appropriate enzymes, the molecules synthesized on the solid phase. Numerous enzyme systems are known and commercially available for this purpose. Examples thereof are DNA polymerases, thermostable DNA polymerases, reverse transcriptases and RNA polymerases.

The reaction products are notable for a great diversity of sequence, which can be programmed in a freely selectable manner indirectly via the template molecules during the preceding synthesis process. A microarray from the Geniom system is able to synthesize in one channel as reaction chamber 6000 freely selectable oligonucleotides having a sequence of up to 30 nucleotides. Accordingly, after the copying step, 6000 freely programmable 30-mer DNA or RNA are present in solution and can be provided as reactants in a next method step or as final product.

It may in this connection be necessary in some embodiments for the start of the copying step to add so-called primer molecules which serve as initiation point for polymerases. These primers may consist of DNA, RNA, a hybrid of the two or of modified bases. The use of nucleic acid analogues such as PNA or LNA molecules as example is also provided in certain embodiments. To create a recognition site for the primer, it may be expedient to add a uniform sequence on the end of each nucleic acid polymer on the support, either as part of the synthesis or in an additional step by means of an enzymatic reaction such as a ligation of a previously made nucleic acid cassette. In one variant, the distal end of the sequence synthesized on the support is self-complementary and is thus able to form a hybrid double strand which is recognized as initiation point by the polymerases.

The purpose of the method is to provide nucleic acids with high and rationally programmable diversity of the sequences for methods following in a next step.

Examples of these methods are:
the preparation of primers for primer extension methods, strand displacement amplification, polymerase chain reaction, site directed mutagenesis or rolling circle amplification,
gene expression modulation by means of RNAi or antisense methods,
preparation or provision of analytes (sample preparation) for logically subsequent analysis by microarrays, sequencing methods, amplification methods (strand displacement amplification, polymerase chain reaction or rolling circle amplification) or analysis in a gel electrophoresis,
RNA libraries for translation in vitro or in vivo,
cloning of sequences by means of vectors or plasmids,
ligation of the nucleic acids into vectors or plasmids,
validation or testing of hybridization assays and relevant reagents and kits by means of the generated nucleic acid polymers in the areas of microarrays, dot blots, Southern or Northern blots, bead arrays, serial analysis of gene expression [SAGE],
reference or calibration methods or method steps within assays from the areas of microarrays, dot blots, Southern or Northern blots, bead arrays, serial analysis of gene expression [SAGE].

The use of nucleic acids as hybridizable reagent is common to all these methods. In addition, there are also methods using nucleic acid polymers not at all or not exclusively via a hybridization reaction. These include aptamers and ribozymes.

The preparation of the nucleic acid polymers provides, at several points in the method, the possibility of introducing modifications or labels into the reaction products by known methods. These include labeled nucleotides which are modified for example with haptens or optical markers such as fluorophores and luminescent markers, labeled primers or nucleic acid analogues with particular properties such as, for example, particular melting temperature or accessibility for enzymes.

Example of a preferred embodiment of the invention and outline of the method:
1. Preparation of a microarray of 6000 different 30-mers in a Geniom® one apparatus with distal 3′ end.
2. Attachment of a generic primer sequence of 15 bases by wet-chemical instead of light-controlled synthesis on all 6000 oligos except a control. The primer sequence is chosen so that a PCR reaction is possible.
3. Processing of the array as far as a state ready for hybridization.
4. Addition of primer and nucleotides, and Taq DNA polymerase and carrying out the PCR reaction cyclically either directly in the Geniom® one apparatus or in a holder for in situ hybridization in another commercially available PCR machine, which does not, however, proceed into the exponential phase.
5. Running through 10 cycles and correspondingly 10-fold copying of the attached template oligonucleotides and a final heating step in order to detach the last second strand.
6. Elution of the copies into an Eppendorf vessel. The vessel now contains 6000 different programmed DNA 45-mers which are available for any desired applications.

### 1.5 EMBODIMENTS, VARIANTS AND APPLICATIONS

### 1.5.1 Reaction Supports and Solid Phases

It is generally possible to use for the method of the invention all reaction supports and solid phases for which synthesis of a matrix of nucleic acid polymers as template of the copying process is established.

These include as representative examples the following reaction support formats and solid phases known to the skilled worker:
flat reaction support, also called “chip”,
porous support,
reaction support with electrodes,
reaction support with temporarily or permanently immobilized solid phase composed of particles or beads,
microfluidic reaction support,
surface modification; gels, linkers, spacers, polymers, amorphous layers, 3D matrices.

Some of these reaction supports can be used in combination, e.g. a microfluidic reaction support with porous surfaces.

### 1.5.2 Preferred Embodiments of the In Situ Synthesis of the Array

Assembly of the DNA probes takes place by light-controlled in situ synthesis in a Geniom® one instrument from Febit using modern protective group chemistry in a three-dimensional microstructure as reaction support. In a cyclic synthetic process, illuminations and condensations of the nucleotides alternate until the desired DNA sequence has been completely assembled at each position of the array in the microchannels. It is possible in this way to prepare up to 48 000 oligonucleotides having a length of up to 60 individual building blocks. The oligonucleotides are in this case covalently bonded to a spacer molecule, a chemical spacer on the glass surface of the reaction support. The synthesis proceeds under software control and makes great flexibility possible in the assembly of the array, which the user can thus configure individually in accordance with his needs. Thus, for example, the length of the oligonucleotides, the number of generated nucleic acid probes or internal controls can be adapted optimally for the particular experiment.

The copying reaction relies on a primer sequence, which matches a primer with a length of 15 bases and has been assembled by a uniform synthesis taking place equally on all the oligonucleotides by means of standard DMT protective group chemistry, distally on the probes. The reaction support comprises 8 separate reaction chambers which can be used individually and need not, but may, comprise the same array. In this embodiment, 45-mers are synthesized on the surface.

The arrays are ready for hybridization after the synthesis of the template oligonucleotides is complete and the protective groups on the nucleobases have finally been removed.

The reaction support is removed and inserted into a heatable (Peltier element) unit comprising a fluidic connection, valves and a pump (piston pump). This unit serves to partially automate process steps. For the copying reaction, a mixture of primer, biotin-labeled nucleotides, restriction enzyme to introduce single-strand breaks on the primer and DNA polymerase is added. Reaction at 32° C. for 4 hours is followed by a single heating step at 90° C. to stop the reaction and bring about denaturation of all double strands present. Since 45-mer oligonucleotides were used for copying, the nucleic acids now present in solution in the reaction mixture comprise firstly the remaining primers (15-mers) and secondly a set of 45-mers. The 45-mers all comprise the complementary sequence of the primer at the 5′ end, but 30 completely freely selectable bases at the 3′ end.

This base sequence is chosen so that in each case two 45-mers form a primer pair for a reaction which now follows. These primers are both located outside an SNP to be analyzed on a target sequence and have a distance of 1-30 bases.

### 1.5.3 Initiation of the Copying Process on the Template Nucleic Acids

It is possible in principle to use all methods known to the skilled worker for initiating an enzymatic nucleic acid copying process for the initiation on the template nucleic acids, such as those known from the polymerase chain reaction, strand displacement and strand displacement amplification, in vitro replication, transcription, reverse transcription or viral transcription applications (representatives thereof are T7 and SP6).

In one embodiment, a T7 or an SP6 promoter is inserted into some or all of the nucleic acid polymers on the reaction support.

In another embodiment, the array of nucleic acids serves to initiate an isothermal copying reaction. One representative of these methods is the strand displacement reaction. Many variants thereof are known in literature. For this purpose for example a primer which binds to the template polymers at their distal end, and can then be extended in the 3′ direction there, is chosen. All or a certain part of the nucleic acid polymers on the support comprise this primer sequence distally. An enzyme for which the primer comprises a recognition site is next added, so that a single-strand break is induced. The usual procedure for this provides for the use of a restriction nuclease, e.g. N.NBstNB I (obtainable for example from New England Biolabs) which naturally introduces only single-strand breaks (so-called nicks) because it cannot form dimers.

### 1.5.4 Rolling Circle Variant

In a further embodiment of the present invention, double-stranded, circular nucleic acid fragments are provided, with one strand being tethered to the surface of the support and the other strand comprising a self-priming 3′ end, so that elongation of the 3′ end is possible. The enzymatic synthesis comprises in this variant of the method of the invention a replication analogous to the rolling circle mechanism known for bacteriophage replication, with one strand of the circular nucleic acid fragment being tethered to the surface of the support and multiple copying thereof being possible. If a double-stranded closed nucleic acid fragment is initially present, the second strand can initially be opened by a single-strand break, forming a 3′ end, starting from which the elongation takes place. The elongated strand can be eliminated for example enzymatically. The partial sequences complementary in each case to the base sequences of the nucleic acid strands tethered to the surface of the support are then synthesized by adding nucleotide building blocks and a suitable enzyme.

### 1.5.5 Labels, Binding Sites and Markers

It is possible in various ways for the products of the copying process to acquire labels, binding sites or markers which are desired for further processing or use in further assays or methods.

These include markers and labels which permit direct detection of the copies and are known to the skilled worker from other methods for copy of nucleic acids. Fluorophores are an example thereof. A further possibility is to provide binding sites for indirect detection methods or purification methods. These include haptens such as biotin or digoxigenin, as examples.

The labels, binding sites or markers may in one variant be introduced by modified nucleotides. A further route is opened up on use of primers for initiating the copying process. The primers may already have label, binding sites or marker when introduced into the reaction.

Labels, binding sites or markers can be introduced subsequently by treating the reaction products of a subsequent labeling reaction with generic agents which react with the nucleic acids. One example thereof are cis-platinum reagents. As an alternative thereto, labels, binding sites or markers can also be introduced by a further enzymatic reaction such as, for example, catalyzed by a terminal transferase.

### 1.5.6 Integration of Sample Preparation and Amplification with an Analytical Microarray

The aim in the embodiments of the invention described here is to integrate sample amplification and sample analysis on one and the same solid-phase support (biochip).

Analysis of DNA and RNA samples has to date required amplification of the sample to be investigated in a first step—both in the construction of gene expression profiles and in genotyping (SNP typing, resequencing etc.). Only in a second step is a highly parallel investigation of the sample to be investigated possible on a biochip via a multiplicity of DNA or RNA receptors. This procedure is time-consuming and costly. This can be solved by the variant described herein according to the method of the invention.

One example of the prior art is described in EP 1 056 884 (method for non-specific amplification of nucleic acids (Van Gemen, PamGene B.V.); inter alia oligo-dT sequence blocked at the 3′ end). Another one is to be found in the publication on “Solid phase transcription chain reaction” or “SP-TCR” of Linden Bioscience.

### 1.5.6.1 Variant 1: Investigation of RNA Analytes Using RNA Polymerase and RnaseH

Exemplary outline of an integrated amplification of one embodiment of integrated sample preparation and analysis of a support with microarray of nucleic acid probes:
specific probes A_{1 . . . n} are assembled for amplifying the target to be investigated, and specific probes B_{1 . . . x} are assembled for analyzing the target to be investigated, on the solid phase.
The probes A_{1 . . . n} comprising the promoter cannot be extended at their 3′ end (either block or solid phase at the 3′ end).
The probes A_{1 . . . n} comprise a promoter sequence (e.g. T3, T7, SP6) which permits amplification by an RNA polymerase of the target sequences to be investigated when there is specific hybridization therewith.
RNase-H cuts the RNA part of the DNA-RNA duplex at the 3′ end; a double-stranded (dsDNA) promoter (T7, Sp6, T3) is subsequently assembled there.
Starting from the dsDNA promoter, the RNA sequences C_{1 . . . y} complementary to the target sequences are prepared in large number (antisense RNA).
The amplified RNA target sequences C_{1 . . . y} can then be analyzed after hybridization onto the specific probes B_{1 . . . x}.

Analysis of the interaction of the target sequences C_{1 . . . y} with the specific probes B_{1 . . . x} is possible by a hybridization-mediated method or else by an enzyme-mediated method.

An RNA polymerase with RNaseH activity (e.g. AMV-RT, MLV-RT) is used for the amplification; mix of rNTPs, dNTPs.

### EXEMPLARY PROCEDURE

The scope of the reaction is broad, and the design has great flexibility in relation to:
orientation of probes A_{1 . . . n}: 5′-3′ or 3′-5′
   orientation of probes B_{1 . . . x}: 3′-5′ or 5′-3′
   nature of the target: RNA (RNA probes would have to be assembled in the case of DNA)
   analytical action principle: enzyme (incorporation of signal emitter during enzymic reaction) or hybridization (incorporation of single emitter during amplification step)

### 1.5.6.2 Variant 2: Investigation of RNA Analytes Using RNA Polymerase

For this purpose, a transcription chain reaction is started in analogy to SP-TCR (see above). To do this, sequence-specific primer sections are combined with RNA polymerase promoters in the template nucleic acids in suitable orientation and taking account of sense/antisense requirements. Well-established representatives of viral RNA polymerase promoters are T7, T3 and SP6. In these cases, the RNA promoter is in each case located proximal to the solid phase, and the sequence-specific section which serves for selective recognition of its complementary section in the target nucleic acids is located distal from the support. Thus, as example, it is possible in an experiment to analyze the mRNA population of an investigated sample in an array of 6000 different DNA oligos (see Geniom® one from Febit) to provide a suitable primer oligo for up to 6000 different sequence sections. If an amplification is to be initiated for each gene, it is possible in this way to prepare amplicons for 6000 genes in parallel in one reaction. By contrast, in a further embodiment, 2 primers are used for each gene, but each comprise one of 2 promoters to be used (e.g. T7 and T3 ). Thus, induction is possible of a gene-specific TCR which prepares 3000 amplicons exponentially for 3000 genes in a single reaction. This reaction product can then be analyzed in any other desired methods. A preferred analysis is a hybridization reaction on a microarray.

In another embodiment, linear or exponential transcription amplification are combined with appropriate analytical probes (as described above).

1.5.6.3 Variant 3: Generation of Sequence-Specific Primers in Solution for Extension Depending on Target Molecules (Target Analyte) in the Sample

In this embodiment, the copies of the template nucleic acids are in turn used for reaction with the target nucleic acids. In this case, the sequences are chosen so that the sequence to be analyzed subsequently in a hybridization reaction is produced only if there is successful extension of the individual copied nucleic acid polymers present in solution. These sections can then in turn be detected by means of an array. In the preferred embodiment, this takes place as described above on by means of analytical probes in the same microarray or on a fluidically connected array.

In one variant for generating the signal, it is possible to provide for the primers for initiating the copying process already to have a modification which assists generation of the signal. One example of such a modification is a primer which has in its 5′ section a branched DNA structure in a region which is not needed for hybridization with the template [Collins M. L. et al.; Nucleic Acids Res. 25(15); 2979-2984; 1997).

Another variant provides for two primers with opposite specificity being provided for each target sequence, i.e., for example, a single gene or exon, so that efficient exponential amplification takes place in a PCR or isothermal amplification.

With simultaneous reaction of copying process, amplification and hybridization onto the analytical probes it is possible in a very compact and simplified format to carry out the complete analysis of a mixture of target nucleic acids. Such a complete analysis can for example clear up the detection of all expressed genes present in a total RNA sample from a biological specimen such as a cell culture population or a tumor biopsy—without previous sample amplification and with very simple sample preparation using standard kits as are available from various manufacturers.

An apparatus belonging thereto consists of
an instrument for the in situ synthesis of the arrays of template polymers and analytical probes,
a detection unit for picking up an optical or electrical signal,
a stored-program unit for controlling the synthesis,
a stored-program unit for controlling the detection and the storage and management of the measured data,
optionally, elements for performing fluidic steps such as sample addition or/and sample discharge,
optionally: elements for automation of sample preparation from biological investigation material, untreated where possible, i.e. for example cell lysis and purification of nucleic acids,
optionally: unit for automated transfer of the prepared sample into or onto the reaction support.

### 1.5.6.4 Signal Generation on Integration of Template-Controlled Amplification with an Analytical Microarray

Examples of signals which can be used in the analysis of the reaction results and of the hybridization onto the nucleic acid polymers provided for this purpose (analytical probes) on the reaction support or array are inter alia the following signals which are well known to experts:

Optical signals
fluorescence (organic and inorganic fluorophores),
light scattering (e.g. gold particles in nm dimensions),
chemiluminescence,
bioluminescence;

Electrical Signals
current,
redox reactions.

The signals can in these cases be introduced into the reaction products by labels, binding sites or markers, similar to those described above. It is moreover possible on the one hand to treat the copies of the template nucleic acids correspondingly. In an alternative embodiment, the labels, binding sites or markers are introduced into the target analytes during a further reaction.

One example thereof is extension of primers which themselves are reaction products of the copying process, depending on target nucleic acids (analytes) in the sample, onto which they can hybridize for this reaction, so that extension occurs only if there is specific hybridization. During this extension, the labels, binding sites or markers are then introduced into these extended polymers so that it is subsequently possible to observe and analyze their binding behavior on the array in connection with the analytical probes.

In a further embodiment, the extended polymers are brought into contact with analytical nucleic acid probes which can in turn be used for extension in the form of a primer extension. The arrangement of a primer extension experiment is known from the specialist literature. The signal of the primer extension onto these analysis probes is then evaluated to determine the result of the analysis. A possible example of such an analysis is determination of single nucleotide polymorphisms (SNPS) in genomic DNA. For this purpose, firstly extendable primers are copied on template nucleic acids. The sequence is chosen so that the SNPs to be investigated are located on the target nucleic acid in the 3′ region downstream of the primer sequence. In the next step, these primers are extended beyond the sequence of SNPs to be detected.

Subsequently, the reaction products of this extension are investigated by primer extension or directly by hybridization, and the results are recorded to determine the SNPs examined in the analysis. The data are processed in the stored-program device for the user of the device according to the invention so that he receives for example directly a report with the base positions and the bases found.

The great advantage of the invention in this connection is that only one universal, generic sample preparation is necessary for such genotyping or SNP analysis assays. Primers and reagents specific for individual genotypes or SNPs are not required, because all sequence specificity is derived from the in situ synthesis of the underlying template arrays and the analysis array. Genotyping and SNP analysis is thus maximally simplified in the embodiment with combination of both these in one reaction support.

### 1.5.7 Validation of Arrays of Nucleic Acids

The use, described at the outset, of nucleic acids and, in certain embodiments, of synthetic oligonucleotides in arrays in which the molecules are disposed as receptors or capture molecules in rows and columns is generally confronted by the very difficult empirical validation of the prepared arrays with the assistance of appropriate sample molecules. This problem is well known to the skilled worker and becomes a problem which is increasingly difficult to solve with the arrangement of several thousand capture molecules in an array. No suitable and expedient validation method is known for developing so-called high-density arrays with more than 100 000 individual reaction chambers. The imperfect solution is to use poorly describable biological samples.

### 1.5.8 Synthetic Genes

In one embodiment, high-quality nucleic acids whose sequence can be programmed freely are provided at low cost and efficiently in the form of oligonucleotides with a length of 10-200 bases in a diversity of 10 or more different sequences in order to prepare synthetic coding double-stranded DNA (synthetic genes).

Assembling double-stranded DNA from oligonucleotides has been known since the 1960s [studies by Khorana and others; see “Shabarova: Advanced Organic Chemistry of Nucleic Acids”, VCH Weinheim]. In most cases it takes place by using one of two methods [see Holowachuk et al., PCR Methods and Applications, Cold Spring Harbor Laboratory Press]:

On the one hand, the complete double strand is synthesized by synthesizing single-stranded nucleic acids (of suitable sequence), annealing these single strands by hybridization of complementary regions and connecting the molecular backbone by enzymes, mostly ligase.

By contrast, another possibility is to synthesize marginally overlapping regions as single-stranded nucleic acids, annealing by hybridization, filling in the single-stranded regions by enzymes (polymerases) and then connecting the molecular backbone by enzymes, mostly ligase.

A preferred outline of a gene synthesis according to the invention is as follows: Synthesis of many individual nucleic acid strands is generally carried out by using the method of the invention for highly parallel template-based DNA synthesis in a modular system. The resulting reaction products are sets of nucleic acids which serve as building blocks in a subsequent process. A sequence matrix which may comprise more than 100 000 different sequences is generated thereby. The nucleic acids are in single-stranded form and can be eluted from the support or be reacted directly in the reaction support. The template can be copied many times, without being damaged, by repeated copying in one or more steps, and at the same time each of the sequences encoded in the matrix is multiplied. As described in detail elsewhere, it is possible by distal-to-proximal copying also to eliminate the content of truncated nucleic acid polymers on the solid phase if the copying initiation site is located distally. One example thereof is a distally attached promoter sequence.

The support with the matrix of solid phase-bound molecules can be stored for renewed use later. The diversity of sequences generated in a reaction support by in situ synthesis is thus made available in an efficient manner for further process steps. It is possible at the same time through the design of the copying reaction to achieve a high quality of the copied sequences.

Suitable combinations of the detached DNA strands are then formed. Joining the single-stranded building blocks to give double-stranded building blocks takes place inside a reaction chamber which may, in a simple approach, be a conventional reaction vessel, e.g. a plastic tube. In another preferred embodiment, the reaction chamber is part of the reaction support which, in one variant, may be a microfluidic reaction support in which the required reactions take place. A further advantage of an integrated microfluidic reaction support is the possibility of integrating further process steps such as, for example, a quality control by optical analysis. In one embodiment, the synthesis of the matrix itself has taken place in a microfluidic support which can then be used at the same time as reaction chamber for the subsequent joining.

The sequence of the individual building blocks is chosen in this case so that, when the individual building blocks are brought into contact, mutually complementary regions are available at the two ends brought together, in order to enable specific annealing of DNA strands through hybridization of these regions. Longer DNA hybrids are produced thereby. The phosphodiester backbone of the DNA molecule is closed by ligases. If the sequences are chosen so that single-stranded gaps exist in these hybrids, these gaps are filled in enzymatically in a known procedure using polymerases (e.g. Klenow fragment or Sequenase). This results in longer double-stranded DNA molecules. Should it be necessary, for further use, to provide these extended DNA strands as single strands, this can take place by methods known to the skilled worker for melting DNA double strands, such as, for example, temperature or alkali.

It is possible by putting together clusters of DNA strands synthesized in this way inside reaction chambers in turn to generate longer partial sequences of the final DNA molecule. This can take place stepwise, and the partial sequences are thus combined to give DNA molecules of increasing length. It is possible in this way to generate very long DNA sequences as completely synthetic molecule having a length of more than 100 000 base pairs. This corresponds to the size range of a bacterial artificial chromosome BAC. 10 000 individual building blocks are required to assemble a sequence of 100 000 base pairs from overlapping building blocks 20 nucleotides long.

This can be done using most of the highly parallel synthetic methods described at the outset. The technologies particularly preferred in this connection for the method of the invention are those which generate the array of nucleic acid polymers in a substantially freely programmable manner and do not depend on the installation of technical components such as, for example, photolithographic masks. Accordingly, particularly preferred embodiments are built on projector-based light-directed synthesis, indirect projector-based light-controlled synthesis using photoacids and reaction chambers in a microfluidic reaction support, electronically induced synthesis by means of spatially resolved deprotection at individual electrodes on the support and fluidic synthesis by means of spatially resolved deposition of the activated synthesis monomers.

For expedient processing of genetic molecules and systematic acquisition of all possible variants it is necessary to prepare the building blocks flexibly and economically in their individual sequence. This is done by the method through the use of a programmable light source matrix for the light-dependent spatially resolved in situ synthesis of the DNA strands which are used as building blocks. This flexible synthesis permits unrestricted programming of the individual sequences of the building blocks and thus also the generation of any desired variants of the partial sequences or of the final sequence, without this being associated with substantial changes in components of the system (hardware). The diversity of genetic elements can be systematically processed only through this programmed synthesis of the building blocks and thus of the final synthetic products. At the same time, the use of computer-controlled programmable synthesis permits the overall process to be automated, including communication with appropriate databases.

The sequence of the individual building blocks can be selected if the target sequence is specified, expediently taking account of biochemical and functional parameters. In this connection, an algorithm searches for suitable overlapping regions after input of the target sequence (e.g. from a database). Different numbers of partial sequences can be constructed, depending on the objective, specifically within one reaction support to be illuminated or distributed over a plurality of reaction supports. The annealing conditions for forming hybrids, such as, for example, temperature, salt concentration etc., are adjusted by an appropriate algorithm to suit the overlapping regions available. Maximum specificity of annealing is ensured in this way. In a completely automatic version, the data for the target sequence can also be taken directly from public or private databases and be converted into appropriate target sequences. The resulting products can in turn optionally be fed into appropriately automated procedures, e.g. into the cloning in suitable target cells.

Stepwise assembly by synthesis of the individual DNA strands in reaction zones inside circumscribed reaction chambers also permits difficult sequences to be assembled, e.g. those with internal repetitions of sequence sections, like those occurring for example in retroviruses and corresponding retroviral vectors. Synthesis of any desired sequence is possible due to the detachment of the building blocks inside the fluidic reaction chambers, without problems arising through the location of the overlapping regions on the individual building blocks.

The high quality requirements necessary for assembling very long DNA molecules are satisfied inter alia through the use of real-time quality controls. This entails monitoring of the spatially resolved synthesis of the building blocks, as well as of the detachment and the joining until the final sequence is produced. All the processes then take place in a transparent reaction support. It is further made possible to follow reactions and fluidic processes in transmitted light by, for example, CCD detection.

The miniaturized reaction support is designed so that a detachment process is possible in the individual reaction chambers, and thus the synthesized DNA strands on the reaction zones located inside these reaction chambers can be detached in clusters. With a suitable design of the reaction support, the joining of the building blocks is possible in a stepwise process in reaction chambers, as is the removal of building blocks, partial sequences or the final product, or else the sorting or fractionating of the molecules.

The target sequence can, after it has been made, be introduced as integrated genetic element by transfer into cells and thus cloned, and be investigated in the course of functional studies. A further possibility is for the synthetic product first to be purified further or analyzed, this analysis possibly being for example a sequencing. The sequencing process can also start through direct coupling to an appropriate instrument, e.g. to an apparatus operating according to the in DE patent application 199 24 327 for integrated synthesis and analysis of polymers. It is likewise conceivable to isolate and analyze the generated target sequences after cloning.

The method of the invention provides, via the integrated genetic elements generated therewith, a tool which acquires the biological diversity for further development of molecular biology in a systematic process. The generation of DNA molecules having desired genetic information is thus no longer the restrictive factor on studies in molecular biology, because all molecules, from small plasmids via complex vectors to minichromosomes, can be generated synthetically and are available for further studies.

The preparation method allows parallel generation of numerous nucleic acid molecules and thus a systematic approach to questions relating to regulatory elements, DNA binding sites for regulators, signal cascades, receptors, effect and interactions of growth factors etc.

It is possible through the integration of genetic elements into a fully synthetic total nucleic acid for the known genetic tools such as plasmids and vectors to be used, and it is possible in this way to build on corresponding experience. On the other hand, this experience will be rapidly changed through the desired optimization of the available vectors etc. The mechanisms which, for example, make a plasmid suitable for propagation in a particular cell type can for the first time be investigated rationally on the basis of the method of the invention.

The entire scope for combination of genetic elements can be opened by this rational investigation of large numbers of variants. Thus, the programmed synthesis of integrated genetic elements is created as second important element besides the highly parallel analytical methods (inter alia on DNA arrays or DNA chips) which are currently undergoing rapid development. The basis for rational molecular biology can be formed only by the two elements together.

In the programmed synthesis of appropriate DNA molecules, not only is any desired composition of coding sequences and functional elements possible, but also adaptation of the intermediate regions. This ought to lead rapidly to minimal vectors and minimal genomes, whereby advantages arise in turn through the smaller size. Transfer vehicles such as, for example, viral vectors can thus be made more efficient, e.g. on use of retroviral or adenoviral vectors.

Beyond the combination of known genetic sequences, it is also possible to develop new genetic elements, which can build on the function of available ones. The flexibility of the system is of enormous value particularly for such development work.

The synthetic DNA molecules are moreover completely compatible, at every stage of development of the method described herein, with available recombinant technology. Integrated genetic elements can also be provided for “traditional” molecular biology applications, e.g. through appropriate vectors. The incorporation of appropriate cleavage sites even for enzymes which have been used little to date is not a limiting factor with integrated genetic elements.

This method makes it possible to integrate all desired functional elements as “genetic modules”, such as, for example, genes, parts of genes, regulatory elements, viral packaging signals etc., into the synthesized nucleic acid molecule as carrier of genetic information. The advantages arising from this integration are inter alia as follows:

It is possible thereby to develop highly functionally integrated DNA molecules omitting unnecessary DNA regions (minimal genes, minimal genomes).

Unrestricted combination of genetic elements, and alterations in the sequence, such as, for example, for adaptation to the expressing organism/cell type (codon usage), are made possible, as are also alterations in the sequence to optimize functional genetic parameters such as, for example, gene regulation.

Alterations in the sequence to optimize functional parameters of the transcript are also made possible, e.g. splicing, regulation at the mRNA level, regulation at the translation level, and moreover the optimization of functional parameters of the gene product, such as, for example, the amino acid sequence (e.g. antibodies, growth factors, receptors, channels, pores, transporters, etc.).

It is additionally possible to produce constructs which intervene in gene expression via the RNAi mechanism. If such constructs code for more than one RNAi species, a plurality of genes can be inhibited simultaneously in a multiplex approach.

Overall, the system implemented with the method is extremely flexible and permits, in a manner which has not previously existed, the programmed production of genetic material with a greatly reduced expenditure of time, materials and work.

Targeted manipulation of larger DNA molecules such as, for example, chromosomes of several hundred kbp was virtually impossible with available methods. Even the more complex (i.e. larger) viral genomes with more than 30 kbp (e.g. adenoviruses) are difficult to handle and manipulate with conventional genetic engineering methods.

There is a considerable shortening up to the last stage of cloning of a gene: the gene or the genes are synthesized as DNA molecule and then (after suitable preparation, such as purification etc.) introduced directly into target cells, and the result is studied. The multistage cloning process, usually proceeding via microorganisms such as E. coli (e.g. DNA isolation, purification, analysis, recombination, cloning into bacteria, isolation, analysis, etc.), is thus reduced to the final transfer of the DNA molecule into the ultimate effector cells. In the case of synthetically prepared genes or gene fragments, clonal replication in an intermediate host (usually E. coli) is no longer necessary. The risk that the gene product intended for the target cell has a toxic effect on the intermediate host is thus avoided. This is a distinct contrast from the toxicity of some gene products which, on use of conventional plasmid vectors, frequently leads to considerable problems in the cloning of the corresponding nucleic acid fragments.

A further considerable improvement is the shortening in time and the reduction in operations until, after sequencing of genetic material, the potential genes found thereby are verified and cloned as such. Normally the finding of samples of interest, which come into consideration as ORF, is followed by the use of probes (e.g. by means of PCR) to look in cDNA libraries for corresponding clones which, however, need not comprise the entire sequence of the messenger RNA (mRNA) originally used to prepare them (problem of full length clones). In other methods, an antibody is used for searching in an expression gene library (screening). Both methods can be abbreviated greatly with the method of the invention: when a gene sequence determined “in silico” (i.e. after identification of an appropriate pattern in a DNA sequence by the computer) is present, or after decoding of a protein sequence, a corresponding vector with the sequence or variants thereof can be generated directly by programmed synthesis of an integrated genetic element and be introduced into suitable target cells.

The synthesis of DNA molecules of up to several hundred kBP in this way permits viral genomes, e.g. adenoviruses, to be synthesized completely and directly. These are an important tool in basic research (inter alia gene therapy), but are difficult to handle with conventional genetic engineering methods because of the size of their genome (about 40 kbp). Fast and economical generation of variants for optimization in particular is greatly limited thereby. This limitation is eliminated by the method of the invention.

Through the method, integration of the synthesis, detachment of the synthetic products and joining to give a DNA molecule take place in one system. It is possible with Microsystems engineering production methods to integrate all necessary functions and steps in the method up to purification of the final product in a miniaturized reaction support. These may be synthesis zones, detachment zones (clusters), reaction chambers, supply channels, valves, pumps, concentrators, fractionation zones etc.

Plasmids and expression vectors can be directly prepared for sequenced proteins or corresponding partial sequences, and the products can be biochemically and functionally analyzed, e.g. using suitable regulatory elements. The search for clones in a gene library is thus dispensed with. Correspondingly, open reading frames (ORF) from sequencing studies (e.g. human genome project) can be programmed directly into appropriate vectors and be combined with desired genetic elements. Identification of clones, e.g. in by elaborate screening of CDNA libraries, is dispensed with. The flow of information from sequence analysis to function analysis has thus been greatly shortened, since an appropriate vector including the suspected gene can be synthesized and made available on the same day on which an ORF is available through analysis of primary data in the computer.

Compared with conventional solid-phase synthesis for obtaining synthetic DNA, the method of the invention is notable for less expenditure of material. To prepare thousands of different building blocks to generate a complex integrated genetic element with a length of several 100 000 kbp, in appropriately parallelized format and with appropriate miniaturization (see exemplary embodiments), a microfluidic system requires distinctly less starting materials than a conventional automatic solid-phase synthesizer for a single DNA oligomer (on use of a single column). The contrast here is between microliters and the use of milliliters, i.e. a factor of 1000.

Taking account of very recent findings in immunology, the presented method permits an extremely expedient and rapid vaccine design (DNA vaccine).

### 1.5.9 Competitive Assays with Mixture of Nucleic Acid Probes on a Solid Phase and Solution

Competition of solid phase-immobilized probes and short nucleic acids in solution for binding to target nucleic acids can be carried out.

### 1.5.10 Great Preference for Full-Length Nucleic Acids on the Array as Copying Templates

It is possible in principle for the enzymatic copying process to be initiated distally, proximally or along the solid phase-immobilized nucleic acid polymers. An additional aspect emerges on distal initiation: the method then essentially copies only full-length products and thus avoids the potential problem of termination products from the in situ synthesis on the reaction support, which then undergo no amplification and are thus not present in the population of copies in their transcribed form.

### 1.5.11 Improvement in the Proportion of Full-Length Nucleic Acids on the Array by Reverse Reaction

The proportion of full-length nucleic acids can be increased by filling in truncated but correct probes by reverse reaction of the copies of full-length products.

1. A method for preparing a plurality of different synthetic nucleic acids, comprising the steps:
(a) provision of a support with a surface which comprises a plurality of positions at each of which different nucleic acid fragments are present, comprising base sequences which are complementary to the nucleic acids to be prepared,
(b) addition of nucleotide building blocks and of an enzyme which brings about generation of different nucleic acids from the complementary base sequences from (a), and
(c) detachment of the nucleic acids generated in step (b) and, where appropriate, provision for further operations.

2. A method for preparing a nucleic acid double strand, comprising the steps:
(a) provision of a support with a surface which comprises a plurality of positions at each of which different nucleic acid fragments are present, comprising base sequences which are complementary to partial sequences of the nucleic acid double strand to be prepared,
(b) addition of nucleotide building blocks and of an enzyme which brings about generation of partial sequences of the nucleic acid double strand to be prepared from the complementary base sequences from (a), and
(c) assembly of the partial sequences generated in step b) to give the desired nucleic acid strand.

3. The method as claimed in claim 1, characterized in that the support is selected from flat supports, porous supports, reaction supports with electrodes, reaction supports with particles or beads, microfluidic reaction supports which optionally have surface modifications such as gels, linkers, spacers, polymers, amorphous layers or/and 3D matrices, and combinations of the aforementioned supports.

4. The method as claimed in claim 1, characterized in that a microfluidic support is provided.

5. The method as claimed in claim 1, characterized in that the nucleic acid fragments from (a) are generated by spatially resolved in situ synthesis on the support.

6. The method as claimed in claim 5, characterized in that the nucleic acid fragments from (a) are synthesized by spatially or/and time-resolved illumination by means of a programmable light source matrix.

7. The method as claimed in claim 6, characterized in that the spatially or/and time-resolved synthesis takes place in a microfluidic support with one or more fluidic reaction chambers and one or more reaction zones within a fluidic reaction chamber.

8. The method as claimed in claim 2, characterized in that the assembly of the partial sequences in step (c) takes place at least partly in one or more steps on the support.

9. The method as claimed in claim 1, characterized in that the nucleic acid fragments from (a) are chosen so that the nucleic acids or partial sequences formed in step (b) can be joined to give nucleic acid double-stranded hybrids.

10. The method as claimed in claim 1, characterized in that a plurality of nucleic acids or partial sequences which form a strand of the nucleic acid double strand are covalently connected together.

11. The method as claimed in claim 10, characterized in that the covalent connection comprises a treatment with ligase or/and a filling-in of gaps in the strands with DNA polymerase.

12. The method as claimed in claim 1, characterized in that step (b) comprises the addition of at least one primer for each position of the support, the primer being complementary to part of the nucleic acid fragment located at this position and step (b) comprising an elongation of the primer.

13. The method as claimed in claim 1, characterized in that double-stranded nucleic acid fragments are provided in step (a), with at least one strand being tethered to the surface of the support.

14. The method as claimed in claim 13, characterized in that step (b) comprises transcription of double-stranded DNA fragments or/and replication of double-stranded RNA fragments.

15. The method as claimed in claim 1, characterized in that nucleic acid fragments comprising a self-priming 3′ end are provided in step (a), and step (b) comprises elongation of the 3′ end.

16. The method as claimed in claim 15, which comprises elimination of the elongation product.

17. The method as claimed in claim 1, characterized in that double-stranded, circular nucleic acid fragments are provided in step (a), one strand being tethered to the surface of the support, and the other strand comprising a self-priming 3′ end, and step (b) comprising elongation of the 3′ end.

18. The method as claimed in claim 17, which comprises elimination of the elongation product.

19. The method as claimed in claim 1, characterized in that the nucleic acid fragments from (a) are generated by:
provision of capture probes at the positions and
binding of nucleic acid fragments from a fluid
passed over the support to the capture probes, where the capture probes are complementary to partial regions of the nucleic acid fragments.

20. The method as claimed in claim 1, characterized in that recognition sequences for specific interaction with molecules such as proteins, nucleic acids, peptides, drugs, saccharides, lipids, hormones or/and organic compounds are present at one or more positions in the sequence of the nucleic acid or of the nucleic acid double strand.

21. The method as claimed in claim 1, characterized in that the sequence of the nucleic acid or of the nucleic acid double strands is a naturally occurring sequence, a non-naturally occuring sequence or a combination of these two.

22. The method as claimed in claim 1, characterized in that the sequence is taken from a database, from a sequencing experiment or from an apparatus for integrated synthesis and analysis of polymers.

23. The method as claimed in claim 1, characterized in that the nucleotide building blocks may comprise naturally occurring nucleotides, modified nucleotides or mixtures thereof.

24. The method as claimed in claim 1, characterized in that modified nucleotide building blocks are used for labeling and subsequent detection of the nucleic acids or of the joined nucleic acid double strands.

25. The method as claimed in claim 24, characterized in that molecules to be detected in a light-dependent or/and electrochemical manner are used as labeling groups.

26. The use of nucleic acids or nucleic acid double strands prepared by the method as claimed in claim 1 for therapeutic or pharmacological purposes.

27. The use of nucleic acids or nucleic acid double strands prepared by the method as claimed in claim 1 for diagnostic purposes.

28. The use as claimed in claim 26, comprising a transfer into effector cells.

29. The use of nucleic acids or nucleic acid double strands prepared by the process as claimed in claim 1, where they are stabilized, condensed or/and topologically manipulated during a stepwise combination and joining or subsequent thereto.

30. The use as claimed in claim 29 where the stabilization, condensation or/and topological manipulation is effected by functional molecules such as histones or topoisomerases.

31. The use of nucleic acids or nucleic acid double strands prepared by the method as claimed in claim 1 as propagatable cloning vector where the propagatable cloning vector can serve in suitable target cells for transcription, for expression of the transcribed sequence, and where appropriate for the isolation of expressed gene products.
